(19) United States
(12) Patent Application Publication
(10) Pub. No.: US 2011/0076332 A1
(43) Pub. Date: Mar. 31, 2011

(54) Dextran-chitosan based in-situ gelling hydrogels for biomedical applications

(76) Inventors: Xiaojun Yu, Fishers, IN (US); Chandra M. Valmikinathan, Elmwood Park, NJ (US); Yixing Cheng, Jersey City, NJ (US)

(21) Appl. No.: 12/807,123

(22) Filed: Aug. 27, 2010

Related U.S. Application Data
(60) Provisional application No. 61/275,286, filed on Aug. 27, 2009.

(51) Int. Cl. A61K 9/14; A61K 31/722; A01N 43/16; A61P 31/00; A01P 15/00; A61P 19/02

(52) U.S. Cl. 424/488; 514/55

(57) ABSTRACT

A biodegradable hydrogel comprises a water-soluble dextran having aldehyde groups cross-linked with a water-soluble chitosan. Various chemical agents may be encapsulated in the hydrogel or bonded thereto for controlled release. The hydrogel may be applied as a coating to reduce the likelihood of bacterial attachment and biofilm growth; used in tissue engineering applications to prevent tissue ingrowth; or used as a matrix in which cells may proliferate. The components of the hydrogel can be applied sequentially as a spray or by immersion and will gel spontaneously at environmental or physiological temperatures.

### CROSS-REFERENCE TO RELATED APPLICATIONS

The present application claims the benefit of U.S. Provisional Patent Application No. 61/275,286, filed on Aug. 27, 2009, the disclosure of which is incorporated herein by reference in its entirety.

### STATEMENT REGARDING FEDERALLY SPONSORED RESEARCH

Not Applicable

### FIELD OF THE INVENTION

The present invention relates to hydrogels applicable to biofilm-retarding surfaces, food coatings, implant coatings, tissue engineering, and household applications.

### BACKGROUND OF THE INVENTION

Infection associated with orthopedic implants is one of the major reasons for the failure of joint replacement surgeries. Infection from bacterial biofilms can be caused by a pre-existing infection in the body pre-operation or from the surgery, and can arise anytime after the procedure. There are about 200,000 hip implant and 300,000 knee implant surgeries performed in the United States alone each year and about 3% of these implants have to be replaced due to Staphylococcus aureus (S. aureus) bacterial infection or failure of the host to integrate the implant. Corrective surgery for such replacements costs around 1.5 billion U.S. dollars every year. It has been observed by several researchers that the attachment of bacteria on implants occurs within the first 48 hours after surgery, leading to biofilm formation and, therefore, failure of the implant.

Infections can also occur from bacteria residing on hospital attire or equipment, where attachment of bacteria and protein (e.g., from blood or other sources) can occur. Reducing the potential for such attachment would reduce the frequency of infections associated with Staphylococcus epidermidis (S. epi) which grows on skin or open wounds. Approximately, 250,000 cases of infection associated with contaminated catheters, attire and tools for surgery are reported each year in the United States alone.

Different methods for preventing bacterial biofilm attachment and infection are currently being developed. Simple prevention methods include treating patients with antibiotics at very high concentrations. Although seemingly efficient, this method has been shown to have little beneficial effect, as well as being toxic to the liver and spleen.

Recent publications disclose the application of certain types of hydrated polymer-based coatings to prevent bacterial adhesion. However, these coatings cannot uniformly coat surfaces or discourage cell attachment and proliferation. Therefore they are inefficient in preventing bacterial infection and may inhibit integration of host cells. In order to avoid this situation, hydrogel-based systems, usually based on polyethylene glycol (PEG), have been used to coat surfaces to prevent bacterial adhesion. However, the use of such systems also limits the potential for tissue ingrowth.

Chitosan, which is a biodegradable polysaccharide, has been evaluated for several biological applications ranging from tissue engineering to retardation of biofilm formation on surfaces. However, most of these techniques do not use the chitosan as a hydrogel, but instead used freeze-dried chitosan scaffolds. Also, in cases where a hydrogel is used, particularly for tissue engineering applications, the hydrogels that were formed are opaque, and do not allow easy visualization of cells encapsulated inside the scaffolds. Also, such hydrogels are not as effective as PEG-based coatings in retarding biofilm formation on surfaces.

Dextran-based hydrogels have also been formed using freeze-drying techniques, UV-crosslinking or, in some cases, introduction of double bonds that render dextran cross-linkable by UV radiation. However, dextran by itself cannot retard biofilm formation or even bacterial attachment. Also, dextran is generally not an effective scaffold material for tissue engineering owing to its brittle nature and the ease with which it dehydrates.

Some of the standard techniques used to form of chitosan-based hydrogels include the promotion of electrostatic interactions. For example, such hydrogels have been formed by electrostatic interaction with negatively-charged polymers such as polyacrylic acid, hyaluronic acid, or even dextran sulfates that are inherently negatively charged.

Chen et al. (Biomaterials 29 (2008) 3905-3913) describes an in-situ gellable hydrogel composed of N-carboxyethyl chitosan and oxidized dextran that is non-cytotoxic for tissue engineering applications. However, the reported gelling system is only capable of forming a gel at 37° C. (Biomacromolecules 2007 April; 8(4): 1109-1115), making it unfeasible to use such gels in applications outside of the body. Further, Chen et al. describes cell encapsulation as well as promotion of surface attachment of the cells, which, for the reasons stated above, is undesirable. Chen et al. prepared the modified chitosan using acrylic acid, which is a non-biodegradable compound and is cytotoxic when accumulated in the body. So, such modified chitosans may not be safe for long-term use.

### SUMMARY OF THE INVENTION

This present invention comprises a composite hydrogel for biomedical applications, such as providing a coating in implants and protecting against bacterial infection. The hydrogel incorporates chemically-modified dextran and chitosan.

In a first embodiment, the present invention is applied as a coating to reduce the likelihood of bacterial attachment and biofilm growth. In some instances of the first embodiment, the coating is applied to implants, such as orthopedic implants for hip or knee replacement or vascular implants (e.g., stents), for its bacteriostatic properties and to promote host integration through cell attachment, proliferation and differentiation inside the hydrogel. In other instances of the first embodiment, the coating is applied to hospital attire or medical implements to reduce the potential for bacterial growth on their surfaces.

In a second embodiment, the hydrogels are used in tissue engineering applications, to prevent tissue ingrowth from the outside of the hydrogel as well as resist bacterial attachment to the hydrogel surface. In some instances of the second embodiment, the hydrogels are transparent and are used to cover the region surrounding the eye after surgery, allowing for better vision and greater enhanced patient comfort than the opaque plasters that are currently in use. In other instances of the second embodiment, the hydrogels are provided with active agents, such as drugs or growth agents, that are incorporated within the hydrogels and released over time.

In a third embodiment, the hydrogel components are provided in a spray that may be used to uniformly coat surfaces such that the hydrogel forms in situ, rendering the surfaces bacteriostatic and resistant to attachment of proteins. In some instances of the third embodiment, the spray is used to coat produce to prevent spoilage from bacteria, allowing storage of produce for longer periods of time. In other instances of the third embodiment, the spray is used to thinly coat household surfaces including kitchen countertops, bathroom sinks, and numerous other items. This will prevent bacterial attachment to such surfaces for a prolonged period, as compared to products that only kill bacteria at the time they are used.

### BRIEF DESCRIPTION OF FIGURES

The patent or application contains at least one drawing executed in color, which includes a color photograph. Copies of this patent or patent application publication with color drawings will be provided by the Office upon request and payment of the necessary fee.

FIG. 1 is a graph showing the gelation times of dextran-chitosan hydrogels according to an embodiment of the incorporating dextrans having various degrees of oxidation;

FIG. 2 is a graph showing the gelation times of dextran-chitosan hydrogels according to an embodiment of the present invention prepared with various amounts of dextran and chitosan;

FIG. 3 is photograph of a dextran-chitosan hydrogel according to an embodiment of the present invention;

FIG. 4 is a bar chart comparing the gelation (“gelling”) times of a hydrogel according to an embodiment of the present invention at various ratios of a modified dextran (DexCHO) to a chitosan;

FIG. 5A is a photograph at a magnification of 10× of chondrocytes growing within a hydrogel according to an embodiment of the present invention;

FIG. 5B is a photograph at a magnification of 25× of chondrocytes growing on the outside of a hydrogel of the same type as the hydrogel of FIG. 3A;

FIG. 6A is a live/dead image of chondrocytes within a hydrogel according to the present invention;

FIG. 6B is a live/dead image of chondrocytes about 100 microns beneath the surface of a hydrogel of the same type as the hydrogel of FIG. 4A;

FIG. 7 is a graph showing cytotoxicity of dextran-chitosan hydrogels according to an embodiment of the present invention using a first cytotoxicity test method;

FIG. 8 is a graph showing cytotoxicity of dextran-chitosan hydrogels using a second cytotoxicity test method;

FIG. 9 is a bar chart comparing the results of cytotoxicity assays performed on inoculated hydrogels according to the present invention and tissue culture plate controls;

FIG. 10 is a bar chart of assayed cell numbers in hydrogels according to an embodiment of the present invention having different ratios of dextran to chitosan;

FIG. 11 is a graph showing the release of bovine serum albumin (BSA) overtime from dextran-chitosan hydrogels according to an embodiment of the present invention;

FIG. 12 is a graph showing the release of vancomycin overtime from dextran-chitosan hydrogels according to an embodiment of the present invention;

FIG. 13 is a chart of the release of bovine serum albumin (BSA) from hydrogels according to the present invention having different ratios of dextran to chitosan;

FIG. 14 is a bar chart of cell number (MTS) assays comparing cell growth on socks with and without coatings of a hydrogel according to an embodiment of the present invention;

FIG. 15 is a group of photographs showing the effects of a hydrogel coating according to an embodiment of the present invention on bacterial growth on socks, wherein photographs labeled “A” and “B” respectively show cotton-based socks and nylon socks, and photographs labeled “1”, “2” and “3” respectively show non-inoculated socks without a hydrogel coating, inoculated socks without a hydrogel coating, and inoculated socks with a hydrogel coating, all of which have been subjected to an MTS assay;

FIG. 16 is a group of photographs showing the effects of a hydrogel coating according to an embodiment of the present invention on bacterial growth on socks, wherein photographs labeled “A” and “B” respectively show cotton-based socks and nylon socks, and photographs labeled “1”, “2” and “3” respectively show non-inoculated socks without a hydrogel coating, inoculated socks without a hydrogel coating, and inoculated socks with a hydrogel coating, wherein the socks are stained with methylene blue;

FIG. 17 is a bar chart of bacteria number estimated by MTS assays comparing growth on tissue culture plates coated with different ratios of dextran-chitosan hydrogel compositions according to an embodiment of the present invention, uncoated surfaces, and thin films of chitosan, as well as inclusion of proteins in selected hydrogels;

FIG. 18 is a graph showing in vitro swelling overtime of a cross-linked hydrogel according to an embodiment of the present invention; and

FIG. 19 is a graph showing in vitro degradation overtime of a cross-linked hydrogel according to an embodiment of the present invention.

### DETAILED DESCRIPTION OF THE INVENTION

Embodiments of the present invention include hydrogels that comprise dextran and chitosan, which may be chemically modified as needed for specific applications. Such hydrogels have the property of being self-gelling, allowing in-situ formation of hydrogels within 3-10 minutes, depending on the ratio of dextran to chitosan. Therefore, surfaces can be uniformly coated with solutions of the hydrogel components which will then gel quickly to form a barrier coating. Once the coating has formed, the hydrogel discourages cells, proteins and bacteria from attaching to the coated surface, thus retarding biofilm formation. However, when cells are added to the solutions prior to gelling, along with an appropriate growth medium, they are encapsulated into the hydrogel and, therefore, stay alive and aid in the production of a matrix for host integration and tissue re-growth. Another major advantage of the system is that it involves an in-situ gelling mixture, which, if processed prior to gelling, can be molded or extruded to form plugs, tubes or other shapes. Once gel formation is complete, the hydrogel exhibits good mechanical stability. Further, dextran-chitosan hydrogels of the present invention may form at all ambient environmental temperatures, in contrast to prior art gels that form only at temperatures near 37° C. Thus, the hydrogels of the present invention can be used in many applications outside of the human body.

Hydrogels made according to embodiments of the present invention have various functional groups that aid binding of proteins, such as fibronectin, which promote attachment of cells and growth of cellular matrix inside of the hydrogels. Hydrogels made according to embodiments of the present invention can also be loaded with growth factors or bacteriostatic proteins, such as bone morphogenic proteins (BMPs) and vancomycin, which will then be released in a controlled fashion. Since the chitosan and dextran from which some of the hydrogels of the present invention are made are natural materials, they have very low immunogenicity and very high biocompatibility.

The examples presented herein describe the formation and characterization of hydrogels made according to an embodiment of the present invention. These examples describe representative embodiments of the invention and are in no way intended to limit the range of embodiments encompassed by the present disclosure. A person skilled in the relevant arts may make many variations and modifications of the hydrogels discussed herein without departing from the spirit and scope of the invention.

Hydrogel Formulations: The selection of hydrogel formulations, according to embodiments of the present invention, depends on multiple factors, including: the degree of chemical modification of the hydrogel components (i.e., dextran and chitosan), concentration of the components in the aqueous solution, and ratios of the two components. The usefulness of the hydrogel formulation is mainly characterized by its gelation time which is the time required to form a solid, complete hydrogel after mixing the two hydrogel components.

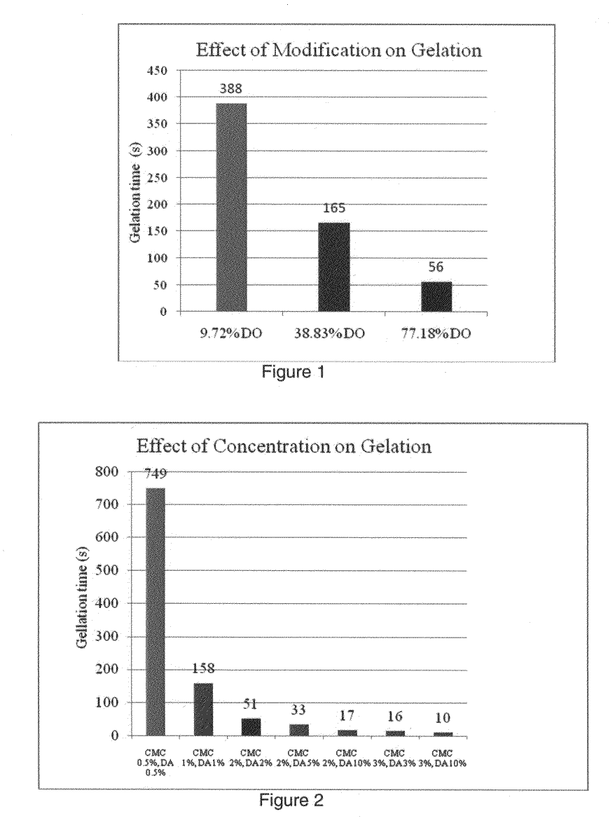

The degree of chemical modification of each of the hydrogel precursors is responsible for the characteristics of the hydrogel. Exemplary chemical modifications of chitosan include deacetylation; for dextrans, an exemplary modification is oxidation of the reactive groups on the polymer. Chitosan having different degrees of deacetylation are commercially available, with a common range from 50% to 100% deacetylation. A series of degrees of oxidization of dextran to dextran aldehyde (DA) can be prepared by varying the amount of oxidizing agent used in the oxidizing reaction. Through a standard trinitrobenzene sulfonic acid (TNBS) assay, DA with degrees of oxidization ranging from 10%-80% have been prepared. Experiments suggest that carboxymethyl chitosan (CMC) with a degree of deacetylation from 50% to 100%, more preferable from 70% to 90%, are useful in forming hydrogels according to the present invention. Experiments also suggest that DA with degrees of oxidization ranging from 10% to 80%, more preferably from 50% to 80% are useful in forming hydrogels according to the present invention. Generally, the higher extents of modification, which in turn lead to higher densities of crosslinking, will lead to faster gelation. In an experiment, a chitosan component with 75-85% deacetylation at constant concentration formed hydrogels with a series of DA having different degrees of oxidization in a 1% solution (w/v). Gelation times were calculated to determine how the degree of oxidization affected the gelation process. Results are shown in FIGS. 1 and 2.

The concentrations of both aqueous hydrogel precursors also greatly influence the gelation process. Higher concentrations imply higher densities of macromers in a certain volume of solution, which in turn implies higher densities of crosslinking between the dextran and chitosan macromers. In a series of experiments, freeze-dried CMC macromer was rehydrated in PBS to reconstitute CMC solutions with concentrations ranging from 0.5% w/v to 4.0% w/v, and freeze-dried DA macromers were rehydrated in PBS to reconstitute DA solutions with concentrations ranging from 0.5% w/v to 10.0% w/v. Solutions of CMC and DA at different concentrations were mixed, and their gelation times were recorded. Generally, higher concentrations of DA and/or CMC lead to faster gelation, with CMC solution contributing more to gelation.

The ratio of DA and CMC plays an important role in determining the gelation time of hydrogels according to the present invention. In a preferred embodiment, CMC and DA were prepared as a 2% w/v solution separately. Different ratios between CMC and DA were used to formulate hydrogel. Generally, hydrogels can be formed in a range of CMC:DA 9:1 to CMC:DA 1:9, more preferably in a range of CMC:DA 7:3 to CMC:DA 3:7. Any ratios within this range will lead to the formation of a hydrogel according to the present invention.

Other chitosan than CMC can be used, but they are not usually water soluble and the acids used for dissolution might have an adverse effect when it comes in contact with human tissues. Other dextrans can be used, but they typically should be aldehyde functionalized. In fact, any large molecule with the hydroxyl group converted to aldehyde can be used to form a hydrogel with modified hydrogels. Depending on the molecular weight, the solution concentrations will have to be adjusted. The concentrations of the component play a role in the consistency of the gels, loading and release of drugs and other similar effects of the hydrogels of the present invention.

A CMC-DA system can be used as a fundamental hydrogel system according to the present invention with further chemical modification being possible. For example, introducing other small molecules or polymers can confer new physicochemical or biological properties to the hydrogel that are absent in hydrogels of CMC and DA alone. Therefore the CMC and DA provide a versatile hydrogel system which allows further modification by taking advantage of their high reactivities.

Since both CMC and DA components have reactive functional groups on their backbones, further chemical modifications are possible on each of the two components. The further modifications on the original CMC and DA components are primarily, but not limited to, covalent bond formation, through, preferably, but not limited to, EDC coupling, DCC coupling, or Schiff base formation. Other modifications based on electrostatic interaction are also possible. These modifications on either CMC or DA introduce small molecules or polymers having reactive functional groups, for example, but not limited to, amino groups, carboxyl groups, hydroxyl groups, thiol groups.

Other amino-containing molecule such as, amino acids, peptide, antibody, 3-amino-9-ethylcarbazole, 4-aminophthalhydrazide, trizma base, rhodamine, cystathionine, luminal, amino-terminated PEI, other aldehyde containing molecules such as, glutaraldehyde, phthaldialdehyde, dodecyl aldehyde, lauric aldehyde, tiglic aldehyde, formaldehyde, resorufin, dexamethasone, other carboxyl-containing molecules such as, amino acid, peptide, acrylic acid, methylacrylic acid, ascorbic acid, anthranilic acid, acid-terminated PEG, decanoic acid, quinic acid, and other reactive molecule such as, FITC, allyl isothiocyanate, 2-hydroxyethyl acrylate, 2-hydroxyethyl methylacrylate, thiocholesterol, 1-thioglycerol can also be used to form hydrogels according to the present inventions, according to the principals discussed above.

Examples of reactive molecules that bind to the DA backbone include: other amino containing molecule such as, amino acids, peptide, antibody, biotin hydrazide, formic hydrazide, benzyl carbazate, 2-hydroxyethyl carbazate, 2-aminoacridone, rhodamine, 2-aminopyridine, amino-terminated polyethyleneglycol (PEG), and polyethylene imides (PEI).

In a further embodiment, any combinations of modified CMC and DA with additional components might lead to the formation of hydrogels conferred with the new property brought by newly introduced molecules, such as increased hydrophilicity or hydrophobicity, increased swelling behavior, fluorescence, radioactivity, all of which might make the CMC-DA system more favorable for a specific biomedical application.

Formation of a Hydrogel: A hydrogel according to an embodiment of the present invention was formed from chitosan and dextran that had been chemically modified to CMC and dextran aldehyde, respectively. CMC was prepared by reacting chitosan in excess sodium hydroxide solution (50% (w/v)) overnight. The alkalized chitosan was collected by vacuum filtration and chloroacetic acid (10 g) dissolved in isopropanol (25 mL) was added drop-wise over a period of 20 min. The reaction was allowed to take place for 6 hrs at 50° C. The mixture was then filtered to remove the solvent and the filtrate was dissolved in water (100 mL). Concentrated HCl was used to adjust the pH to 7. The solution was centrifuged for the removal of the precipitate and the supernatant was added to chilled ethanol (200 mL). The product precipitated from the solution and was collected through vacuum filtration and washed several times using ethanol. The product was vacuum dried at room temperature and dialyzed to remove all excess reagents before further use.

DA was prepared by reacting 2.5% (w/v) dextran in deionized water with 1.65% (w/v) sodium periodate overnight under agitation. The reaction mixture was then quenched with polyethylene glycol (PEG) and dialyzed for one day against deionized water. Solid DA was collected after freeze drying.

Both CMC and DA were separately reconstituted in deionized water at a concentration of 10 mg/ml. To prepare the gels, the DA solution was added to the CMC solution, thoroughly mixed, and stored away from light as this step is a light sensitive process. The resulting hydrogels were then tested as described hereinbelow.

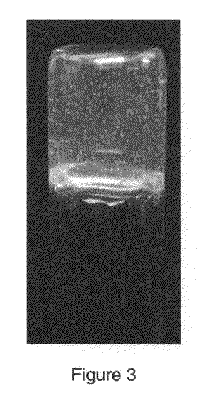

The resulting hydrogels showed good mechanical stability. FIG. 3 shows a transparent hydrogel formed by the method described above. The hydrogel does not flow freely and maintains its shape well. Mechanical properties of the hydrogel can be controlled by altering the concentration ratio between the dextran and chitosan components.

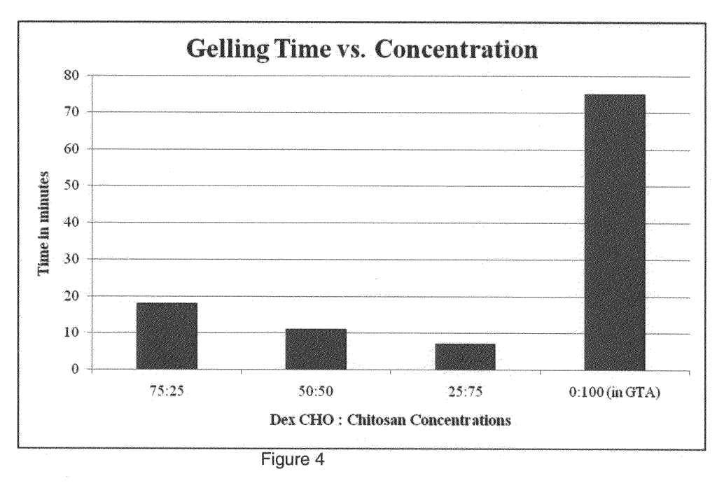

The gelling time for the hydrogels can be controlled by varying the ratio between the dextran and chitosan components. Variations in gelling time at different component ratios are shown in FIG. 4, where “DexCHO” represents DA.

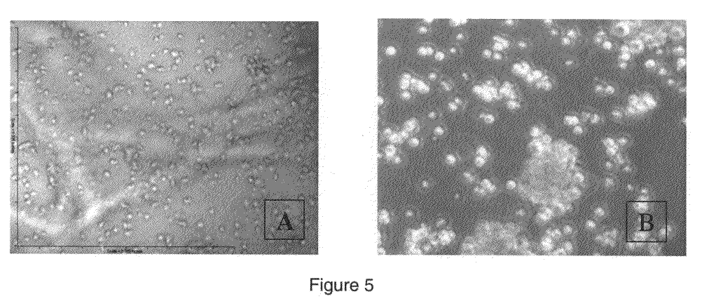

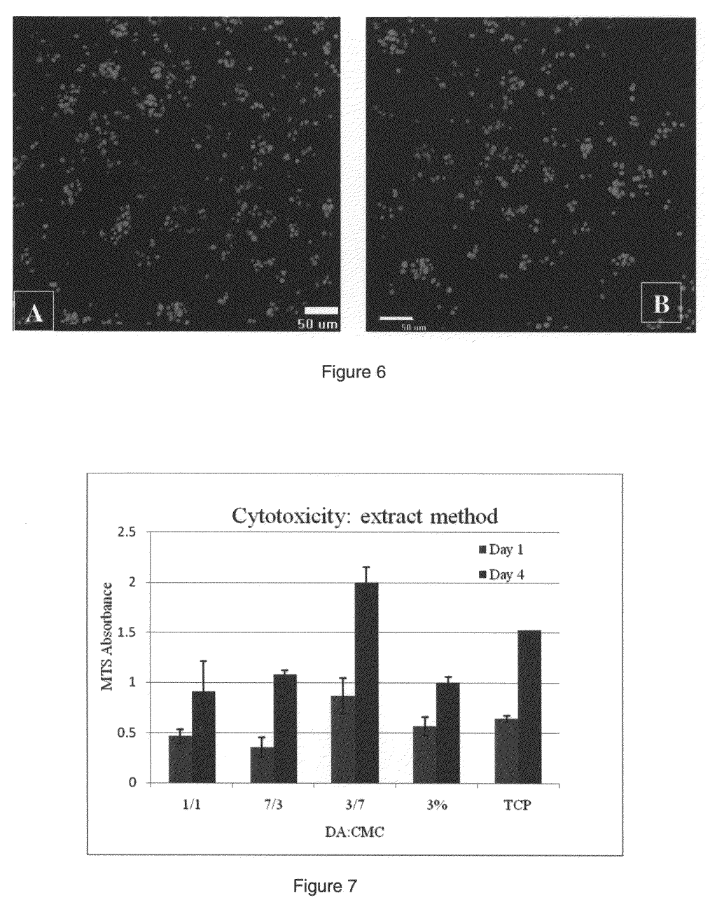

Cell Growth: Chondrocyte growth was investigated to determine whether the DA-CMC hydrogels would allow for cell growth. Chondrocytes (1×10⁵ cells) were seeded within the first of two hydrogels by incorporating them in the DA-CMC mixture before the hydrogel formed. Chondrocytes were seeded on the surface of the second hydrogel. FIG. 5A shows a photograph of chondrocytes growing inside of the first hydrogel at a magnification of 10×. The chondrocytes are distributed throughout the hydrogel. FIG. 5B is a photograph of chondrocytes growing on the surface of the second hydrogel at a magnification of 25×. The chondrocytes are growing normally, but are not attaching to the surface or spreading across it. FIGS. 6A and 6B are live/dead images of the chondrocytes within the first hydrogel, taken with a confocal microscope. FIG. 6A is a Z-stack image of chondrocytes within the hydrogel, and FIG. 6B is an image of chondrocytes about 100 microns beneath the surface of the same type of hydrogel of FIG. 6A. The respective scale bars indicate 50 microns. The green color in both images indicates that most of the cells are indeed alive and growing. Both of the microscopic images indicate that chondrocytes can live normally within the matrix of the hydrogel of the present invention while not attaching to the surface of the hydrogel. Thus, they retain their spherical morphology.

Other cells that can be encapsulated into the hydrogels of the present invention include, but are not limited to, stem cells, marrow cell, bone cells, hepatocytes, keratinocytes, chondrocytes, osteocytes, endothelial cells, epithelial cells, and smooth muscles cells.

MTS Assay Procedure and Cytoxicity Studies: The cytotoxicity of this composition hydrogel has been investigated using osteoblasts as model cells. The results of the cytotoxicity tests show that the hydrogels of the present invention possess non-to-minimal cytotoxicity to osteoblast.

The cytotoxicity of hydrogels according to the present invention is measured in vitro by two methods, namely an extract method and a direct contact method.

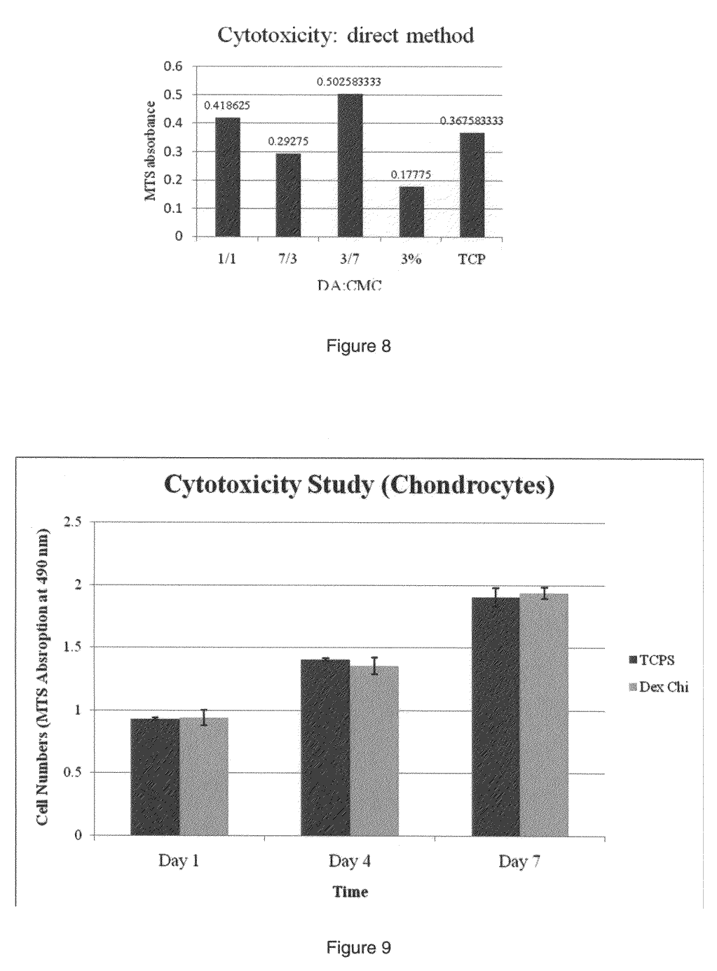

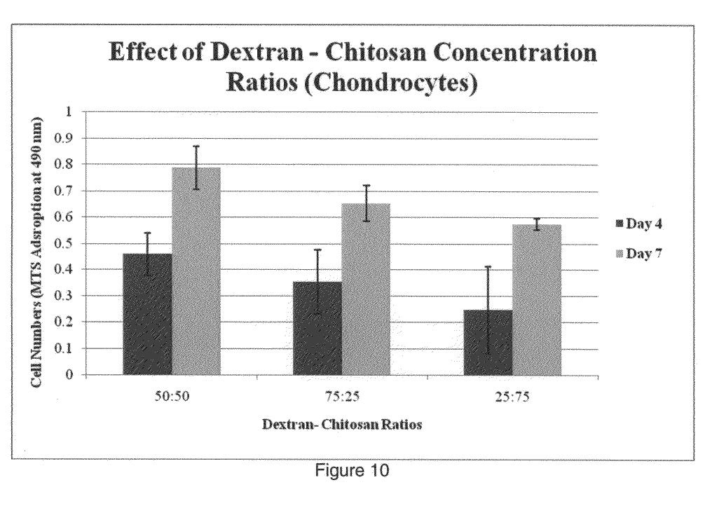

Extract method: The extract procedure was performed according to ISO10993. Hydrogels with volumes of about 1 ml were prepared at various ratios of CMC and DA. Hydrogels were formed in wells of a 24-well plate and allowed to gel for 10 mins in an incubator at 37° C. After incubation, each hydrogel was taken out of the original plate and placed in one well of a 6-well plate. 5 mL of DMEM (90% DMEM, 10% FBS, 1% Penicillin and streptomycin) medium was added to extract the hydrogel under 37° C., 95% humidity and 5% CO₂. 1 ml of hydrogel was extracted to make 5 mL of pretreated media. Osteoblast cells were seeded in wells of a 24-well plate at a density of 4×10⁴ cells per well. After incubation with non-pretreated medium for 24 hours, the medium was discarded and replaced with 1 mL of the aforementioned pretreated medium. Referring to FIG. 7, cell viability was examined at Day 1 and Day 4 using MTS for quantitative measurement.
Direct Method: Hydrogels with various ratios of CMC to DA were prepared in wells of a 24-well plate. The volume of each hydrogel was 1 mL. After incubation for 10 mins at 37° C., each hydrogel was taken out of its well and cut into four pieces. Osteoblasts were seeded in a 24-well plate at a density of 5×10⁴ cells/well in 1 mL of medium and cultured for 24 hours. Previously prepared hydrogels were cut into 4 pieces and each piece was put into one well and incubated with osteoblasts. TCP serves as the control group. Referring to FIG. 8, cell viability was measure at day 1 using a MTS/assay. Before the MTS assay, incubated medium and pieces of hydrogel were discarded and the wells refilled with fresh medium.
Cytotoxicity Study: The cell cytoxicity test ws extened to chondrocytes. Chondrocytes (1×10⁵ cells) were seeded within the hydrogels, and allowed to grow. Cell growth was determined through an MTS assay which was performed in the manner described above. Assays were performed in triplicate at days 1, 4 and 7 in order to study chondrocyte attachment and proliferation. As a control, wells without the hydrogels, or tissue culture plates (TCPS), were used. The hydrogels show no cytotoxicity compared to the control, as shown in FIG. 9.
Effect of Dextran-Chitosan Concentration Ratios on Cell Growth: Three hydrogels were made at DA-CMC concentration ratios of 50:50, 75:25, and 25:75, each of which was prepared with chondrocytes (1×10⁵ cells) before gelling. Cell growth was determined through an MTS assay which was performed in the manner described above. Assays were performed in triplicate at days 4 and 7 in order to study chondrocyte attachment and proliferation. The 50:50 DA-CMC hydrogel showed the greatest cell proliferation of the three hydrogels, as shown in FIG. 10.
Effects of Dextran-Chitosan Concentration Ratios on Drug Release: One or a combination of some bioactive agents can be entrapped in the hydrogel compositions for controlled release. The term bioactive agents describes chemical agents that are introduced into an animal or human subject to produce a biological, therapeutic or pharmacological result. Exemplary bioactive agents which may be introduced according to the present invention to include, for example, angiogenic factors; growth factors; hormones; anticoagulants, for example heparin and chondroitin sulphate; fibrinolytics such as tPA; amino acids; peptides and proteins, including enzymes such as streptokinease, urokinase and elastase; steroidal and non-steroidal anti-inflammatory agents such as hydrocortisone, dexamethasone, prednisolone, methylprednisolone, promethazine, aspirin, ibuprofen, indomethacin, ketoralac; antibiotics, including noxythiolin and other antibiotics to prevent infection; prokinetic agents to promote bowel motility; anti-cancer agents; neurotransmitters; immunological agents including antibodies; nucleic acids including antisense agents; fertility drugs, psychoactive drugs; and local anesthetics.

A wide variety of active agents can be incorporated into the hydrogel. Release of the incorporated additive from the hydrogel is achieved by diffusion of the agent from the hydrogel, degradation of the hydrogel, and/or degradation of a chemical link coupling the agent to the polymer. An “effective amount” refers to the amount of active agent required to obtain the desired effect.

Three significant methods by which active agents can be incorporated into the hydrogel composition are described herein. First, active agents with appropriate functional groups can be conjugated to the backbone of CMC to form an active agent-CMC conjugate which further forms a hydrogel with a DA component. In such an embodiment, dexamethasone is first conjugated to the CMC macromer taking advantage of Schiff base formation chemistry. Then, this dexamethasone-CMC conjugate is mixed with DA, hydrogel appears upon mixing the two components together. In such a case, the release of the active agents conjugated on CMC components greatly relies on the hydrolytic cleavage of the covalent bond that binds the agent to CMC, therefore the diffusion of active agent is subjected to the hydrolysis rate of such a bond and the degradation of the entire polymeric structure, not just the free diffusion of the small active agent. The mechanism of release ensures a more sustainable release than just physically encapsulating active agents in the internal matrix of the hydrogel.

In a second method, active agents with appropriate functional groups can be first conjugated with DA to form an active agent-DA conjugate which further forms a hydrogel upon mixing with a CMC component. In one such embodiment, bovine serum albumin (BSA) is first mixed with a DA solution to allow Schiff base formation between BSA and DA which results in a BSA-DA conjugate. The BSA-DA conjugate is then mixed with CMC to form a hydrogel upon mixing. In such a case, the release of the active agent is subject to the hydrolysis of the covalent bond that binds the agent and DA together and the degradation of the entire polymeric architecture. When compared to free diffusion of other physically encapsulated molecules, this agent-macromer conjugate provides a more sustainable release behavior.

One special property of the active agent-DA conjugate is that the chemical linkage of the agent to the water-soluble polymer can be manipulated to hydrolytically degrade, thereby releasing biologically active agent into the environment in which they are placed. When implanted into a tissue, the controlled-release matrix will release the agent-polymer conjugate, which will release active agent molecules to treat the area of the tissue in the immediate vicinity of the polymer. The agent-polymer conjugates will also diffuse within the tissue, reaching a great distance from the matrix because of their low rate of clearance from the tissue. As the agent-polymer conjugates diffuse, the bond between the polymer and the agent will slowly degrade in a controlled, pre-specified pattern. Other variables which affect conjugate release kinetics are: component ratio, degree of substitution, type of covalent bond, contact surface and so on.

In the third method, non-bonding active agents can be incorporated into the hydrogel by mixing them with the dextran and chitosan. For water soluble active agents, a solution of the agent may be mixed with the dextran and chitosan solutions. For water insoluble active agents, a suspension of the agent may be mixed with the dextran and chitosan solutions. In an embodiment of the present invention, the chemically inert bactericide vancomycin is encapsulated into the hydrogel composition, and the release thereof is controlled by diffusion. Release by diffusion is typically more rapid than the release of covalently-bonded or conjugated compounds. In still a further embodiment, active agents complexed with nanoparticles, micelles, microspheres, liposomes, or other microscale or nanoscale structures can be also loaded into the CMC-DA hydrogel allowing a sustained release of such complexes.

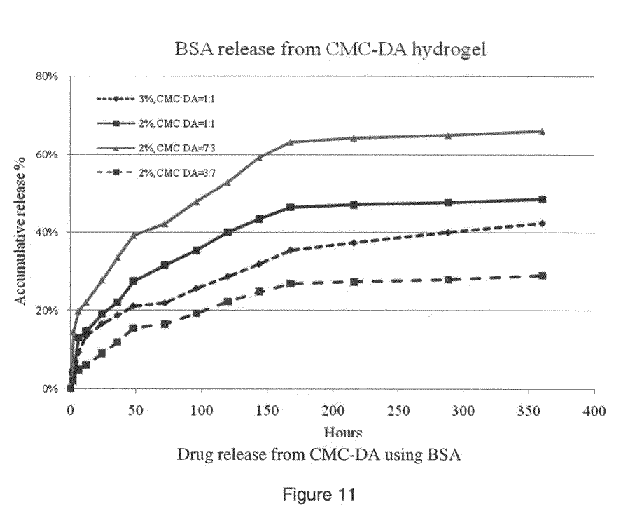

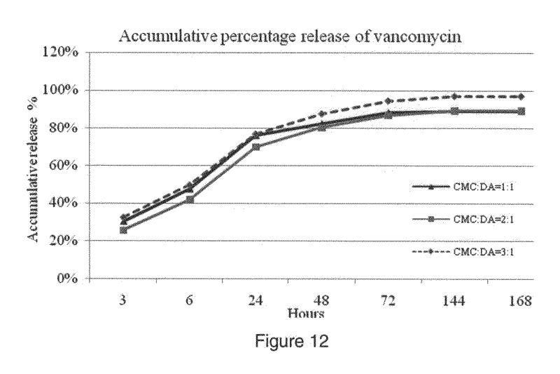

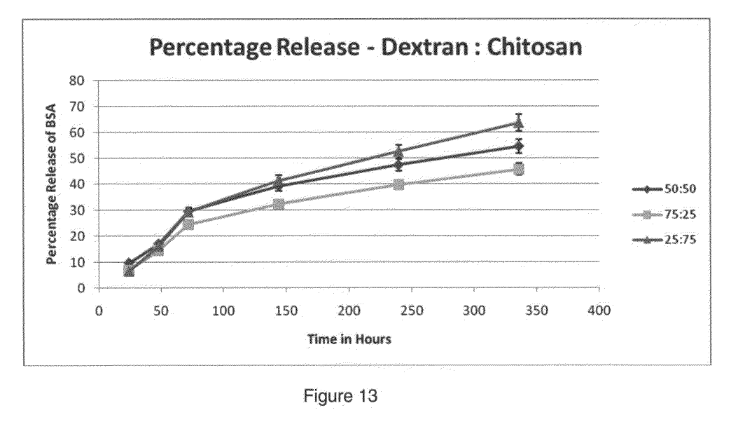

To investigate how the ratio and concentration of dextran and chitosan can vary the release profile of both BSA and vancomycin were tested as models for the release of a protein drug and a hydrophobic drug, respectively. Referring to FIGS. 11 and 12, the results of these tests imply that the ratios, as well as concentrations of the dextran and chitosan affect the hydrodynamic properties of the hydrogel, thus further controlling the release process of the entrapped drugs.

The methods of incorporating bioactive agents described above have been extended to conjugating bioactive agents with hydrogel macromers prior to hydrogel formation. In this way, many bioactive agents containing reactive functional groups can be conjugated to the macromer through a covalent bond that is susceptible to hydrolysis, and then be released through the cleavage of the bond in a sustainable manner. For example, dexamethasone can be first conjugated to the CMC backbone and this dexamethasone-CMC conjugate can be mixed with DA to form a dexamethasone-loaded hydrogel that releases dexamethasone in a sustained manner.

The species of bioactive agents useful with the hydrogels of the present invention are not limited to those mentioned above and can be extended to many other species. In the meantime, the release profile of each individual bioactive agent is not solely dependent on the ratio and characteristic of the hydrogel, but also relies on the hydrophilicity, hydrophobicity and hydrodynamics of the agent itself.

Bovine serum albumin (BSA) was used to show the drug release properties of the DA-CMC hydrogels. Three hydrogels were made at DA-CMC concentration ratios of 50:50, 75:25, and 25:75, each of which was prepared with BSA (2 mg/mL) before gelling. The hydrogels were kept at 37° C. with 1 mL of PBS added to each sample. PBS was periodically collected and replaced with fresh PBS over a 14 day period. The BSA in the collected PBS was quantified using a Bio-Rad™ protein assay kit (Bio-Rad Laboratories, Hercules, Calif.), with absorbance read at 570 nm. At least three gels were sampled at each time point and quantified for the amount of BSA in PBS, with the BSA concentrations reported as the mean concentration±standard deviation. It appears that as DA crosslinks with CMC, it also has the ability to crosslink with BSA itself. Therefore, a higher concentration of DA within the hydrogel causes a lower rate of release of BSA. This trend is clearly illustrated in FIG. 13. Overall, a high rate of drug release is shown for all DA-CMC hydrogels tested.

The BSA was used as a model protein as it has a molecular weight similar to other growth factors and is easily detectable using simple assays. The BSA controlled release results can be extended to all growth factors, proteins and antibacterials.

Determination of Bacteriostatic Properties: Escherichia coli (E. coli) was the bacteria used to study the resistance of the DA-CMC hydrogels to bacterial attachment. Two different types of sock samples, one cotton-based (“A”) and the other nylon (“B”), were used in this study. Each sock was tested in triplicate for each of the following cases: sock sample with no bacteria (“1”); sock sample with bacteria (“2”); and hydrogel-coated sock sample with bacteria (“3”). All of the sock samples were sterilized using 70% ethanol and set up in a 24-well plate. Socks for case 3 were coated with the hydrogel by dipping each sock first in a CMC solution and then in a DA solution. E. coli were cultured in Luria broth (LB broth), and a 500 μl suspension of the culture was added to each of the appropriate wells. Three samples were prepared for each sock type in each case. The samples were incubated overnight to allow for attachment and proliferation of the bacteria.

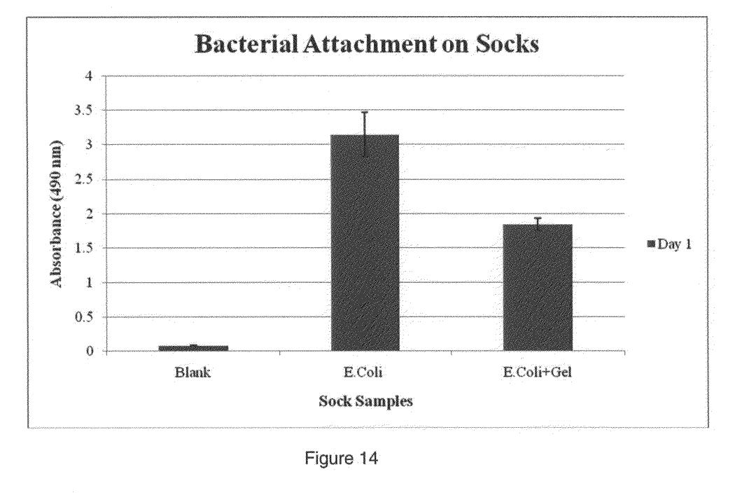

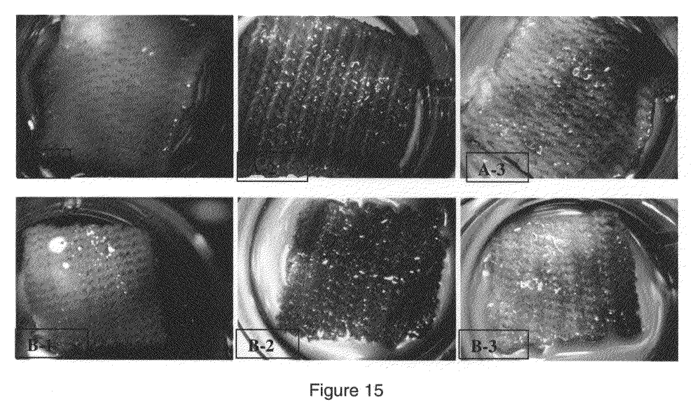

After incubation, an MTS assay was performed on each sock sample using the procedure outline above. The hydrogel-coated samples showed a 50% reduction in bacterial attachment when compared with non-coated inoculated samples, as shown in FIG. 14. Photographs were taken of each sock sample showing the difference in color due to the MTS assay. These photographs are shown in FIG. 15, wherein photographs labeled “A” and “B” respectively show cotton-based sock samples and nylon sock samples, and photographs labeled “1”, “2” and “3” respectively show non-inoculated sock samples without a hydrogel coating, inoculated socks without a hydrogel coating, and inoculated socks with a hydrogel coating.

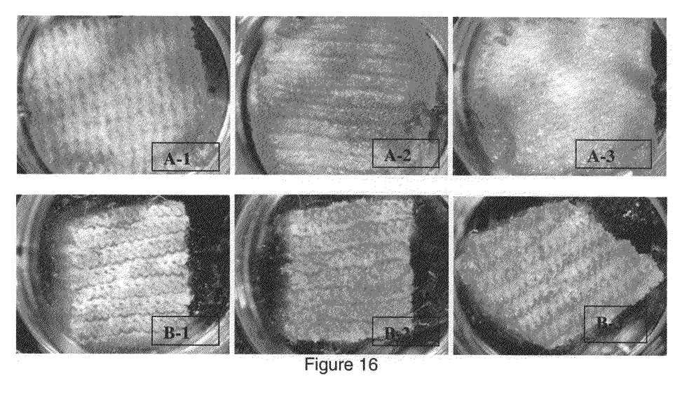

After the MTS assays, the sock samples in the wells were washed with methanol to fix the bacteria, and stained with methylene blue for further quantification. FIG. 16 shows photographs of each type of sock sample, arranged in the same manner as the sock samples of FIG. 16, with the difference in color reflecting the differing degrees of bacterial attachment.

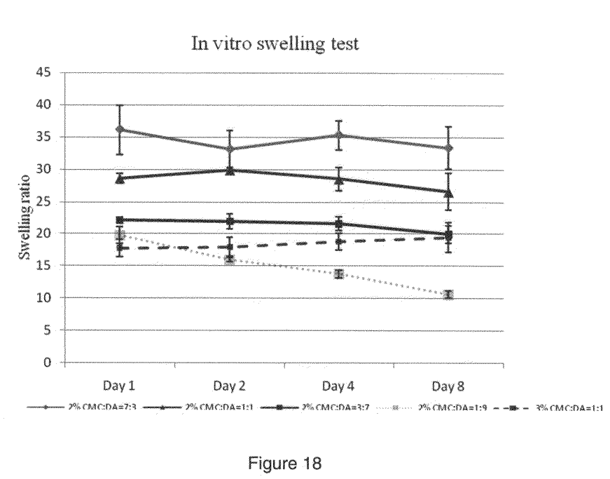

Determination of bacteriostatic action against S. Aureus bacteria: Staphylococcus aureus (S. aureus) was used to study the resistance of selected dextran-chitosan hydrogels of the present invention to bacterial attachment and biofilm formation. Six different types of gels, having different chitosan-to-dextran ratios, proteins and bactericidal drugs, with six replicates each, were prepared in 96-well plates under sterile conditions. FIG. 18 is a bar chart showing the relative extent of bacterial growth, as estimated by MTS assay, for the various cases studied.

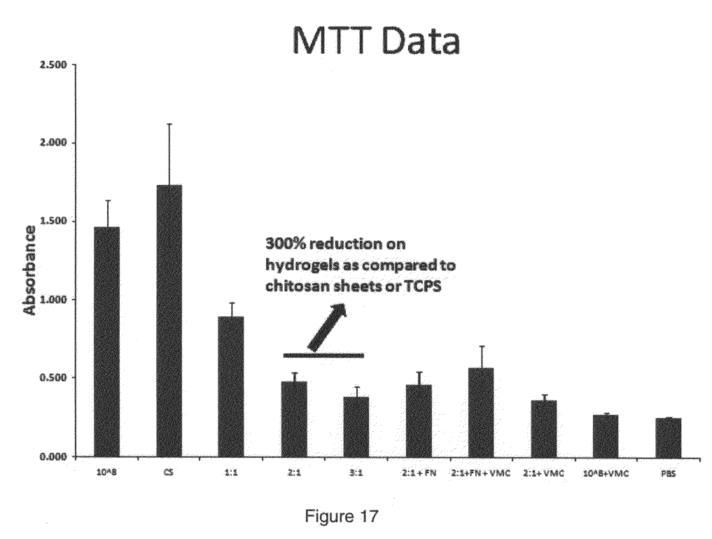

The chitosan and dextran solutions used in this study were prepared as described above. With reference to the labels used in FIG. 17, hydrogels were prepared with chitosan-dextran ratios of 1:1 (“1:1”), 2:1 (“2:1”) and 3:1 (“3:1”). Wells with chitosan sheets alone (“CS”), empty wells (“10̂8”), wells with vancomycin alone (“10̂8+VMC”) and wells with PBS alone (“PBS”) were used as controls. Additional hydrogels having chitosan-dextran ratios of 2:1 were prepared with fibronectin (“2:1+FN”), vancomycin (“2:1+VMC”) or both (“2:1+FN+VMC”) were also studied to understand the effect of drug loading and release on bacterial colony formation.

For the study, triplicate plates of each combination were seeded with S. aureus at a concentration of 1.0×10⁸ bacteria/ml. The bacteria were allowed to attach and grow overnight (16 hours). The same protocol was followed for the MTS assay.

Results for each of the hydrogels showed substantially less bacterial growth and biofilm formation as compared to chitosan based sheets (“CS”) or the tissue culture plate (“10̂8”). It is evident that the hydrogels resisted bacterial attachment. Especially, attachment to the “2:1” and “3:1” hydrogels was significantly less than the “1:1” hydrogels. It is also clearly shown that the addition of fibronectin (“2:1+FN”) did not significantly change bacterial attachment relative to the “2:1” hydrogels. It may be noted that osteoblast cell attachment (results not shown) is significantly changed when fibronectin is added to the hydrogels.

It can also be seen that vancomycin reduced bacterial colony formation, whether or not a hydrogel was present, however some bacterial attachment did occur. However, vancomycin should be steadily released from the hydrogel over time resulting in a sustained bacteriostatic or bacteriocidal effect.

The chitosan and dextran ratios can be altered to alter the mechanical property of the hydrogels. They can be made to very viscous flowable hydrogels or strong hard hydrogels based on the ratios of chitosan and dextran used.

Effects of CMC-DA Ratio's on Hydrogel Properties: The ratio of the two components forming the hydrogel plays an important role in determining the properties of the resulting hydrogel, including gelation time, mechanical strength, swelling behavior, degradation rate, release behavior, cytotoxicity, inhibition of bacterial and epithelial adhesion, and so on. In an embodiment of the present invention, CMC and DA were prepared as a 2% w/v solution separately. Different ratios between CMC and DA were used to formulate the hydrogels. Generally, a hydrogel will form in a range of CMC:DA=9:1 to CMC:DA=1:9, more preferably in a range of CMC:DA=7:3 to CMC:DA=3:7. Any ratios within this range will lead to the formation of a hydrogel.

The effects of CMC-DA ratios on the properties of the resulting hydrogels were characterized in terms of gelation time, mechanical strength, swelling behavior, degradation, and so on, as discussed herein below.

The gelation time of the hydrogel composition can be varied from 5 seconds to as long as 10 minutes, and longer if desired. The gelation time will generally be affected by the ratio of the two components. Generally, a greater proportion of CMC to DA will lead to shorter gelation time. To be useful in most medical applications, the hydrogel should form within one hour after introduction of the mixed components into the mammalian body, as illustrated in FIGS. 1 and 2.

The firmness or mechanical strength of the hydrogel will be also determined in part by the crosslink density between the two components. The maximum crosslink density is obtained by employing the ratio CMC:DA=1:1. Generally, a maximally optimized crosslink between the two components will lead to the toughest mechanic strength of the hydrogel. As the crosslink density deviates from its maximum, the mechanical strength of the hydrogel get weaker.

The swelling of the hydrogel is inversely proportional to the crosslink density which, in turn, is determined by the ratio of dextran and chitosan. Generally, higher crosslink density results in less swelling.

The degradability of the hydrogel is also determined by the ratio of dextran to chitosan. Generally, an optimized ratio will make the hydrogel more stable under physiological conditions and more resistant to hydrolytic cleavage, which leads to slower degradation in the body or in a biomimetic environment.

Mechanical strength: A dynamic material analyzer was employed to test the compressive modulus of a sample hydrogel. Compressive modulus of elasticity was measured in the elastic region of the hydrogels. Sample hydrogels were prepared by incubating the DA and CMC mixture in a 24-well plate for 30 min to obtain columnar hydrogels, typically with a height between 6 to 7.5 mm. Measurements were conducted at 25° C., and a constant strain rate of 0.01× height up to 60% strain was applied to samples.

Mechanical strength of hydrgels with various ratios and concentation

| Concentration | | Compressive | Yield |
|---|---|---|---|
| (% w/v) | Ratio (DA:CMC) | modulus (kPa) | point (kPa) |
| 3% | 5:5 | 22.7664 ± 4.1592 | 5.768 |
| 2% | 7:3 | 5.779255 ± 0.45535 | 1.632 |
| 2% | 5:5 | 8.604 ± 0.41777 | 2.742 |
| 2% | 3:7 | 6.28875 ± 1.037112 | 1.495 |
| 1% | 1:1 | 1.253 ± 0.098517 | 0.372 |

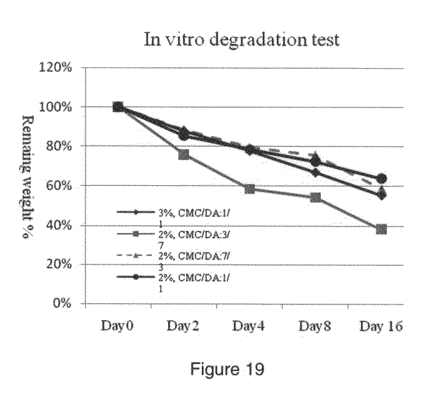

In vitro swelling test: The hydrogels were lyophilized and their dry weights are measured. Dried hydrogel samples made from various ratios of dextran to chitosan were immersed in PBS (pH 7.4) and incubated at 37° C. in order to allow them to reach the swelling equilibrium, each hydrogel occupying a well in a 6-well plate. Every three days, the PBS for incubation was replaced with fresh PBS. At predetermined time intervals, (e.g., days 1, 2, 4, 8, 16 and 24), a swollen hydrogel was taken out of the well. Residual water on the exterior surface of the hydrogel was carefully blotted with paper. FIG. 19 depicts the values obtained during the process which are the average of 4 samples at days 1, 2, 4 and 8. The swelling ratio (Q) was calculated by Q=Wₛ/W_{d}, where Wₛ is the wet weight of the hydrogel and W_{d} is the initial dry weight of the hydrogel.
In vitro degradation test: Biodegradation is a process in which polymeric material depolymerizes or decomposes (e.g., by enzymatic digestion or hydrolytic degradation under physiological conditions), and the resulting small molecules are either absorbed by body or secreted. The prevailing mechanism of degradation is hydrolysis of the hydrolytically unstable polymer backbone. Biodegradability is a desired property for many biomaterials employed in various biomedical applications, such as a scaffold in bone and cartilage engineering, where such scaffolds are temporarily used to support and maintain a specific architecture of cells and need to be absorbed after some extent of regeneration has been achieved. Deposition of non-degradable material can be toxic to the entire body, in which case the implanted material needs to be removed by surgery.

The biodegradability of the hydrogels of the present invention was investigated gravimetrically in PBS (pH 7.4) at 37° C. to mimic physiological conditions. Freeze-dried CMC-DA samples were measured and incubated in PBS. At predetermined times, three samples were taken out of the PBS and weighed after being freeze-dried. Biodegradation was indicated by the weight lost from the hydrogel.

As the results suggested, this CMC-DA hydrogel system degrades in biomimetic environment at an acceptable rate. As shown in FIG. 20, a desired degradation rate can be obtained by varying the ratio and concentration of the two components.

Contrast agent: In some embodiments of the invention, a contrast agent may be included in the hydrogel compositions. A contrast agent is a biocompatible material capable of being monitored by, for example, radiography. Water soluble or water insoluble contrast agents may be used. Examples of water soluble contrast agents include metrizamide, iopamidol, iothalamate sodium, iodomide sodium, and meglumine. Examples of water insoluble contrast agents are tantalum, tantalum oxide, barium sulfate, gold, tungsten, and platinum. These are commonly available as particles preferably having a size of about 10 um or less.

The contrast agent can be loaded to the hydrogel composition prior to administration. Both solid and liquid contrast agents can be simply mixed with dextran and chitosan solutions. Contrast agents are desirably added in an amount of about 10 to 40 weight percent, more preferably about 20 to 40 weight percent.

Biofilm Inhibition: Microrganisms adherent on implant surfaces can grow to form biofilms which are encased in a hydrated matrix of extracellular polymeric substances. These types of biofilms on implants represent a substantial challenge for successful medical treatments and often require implant device removal followed by systemic antimicrobial therapies to clear infections at substantial cost and morbidity. Most often, infection persists until the implant is removed, while the prospects of a revision surgery are lower than those of any primary implant because the surrounding tissue may remain compromised by bacterial presence. In an effort to reduce the incidence of biofilm, a vast number of antiadhesive and/or antimicrobial coatings continue to be reported to minimize microbial adhesion and subsequent biofilm formation on biomaterials surfaces.

Broadly, the biofilm-inhibiting composition coating for a medical device inhibits the growth or proliferation of biofilm microorganism on at least one surface of the medical device. Preferably the device is an implantable device such as drug delivery pump, a pacemaker, a cochlear implant, an analyte sensing device, a catheter, a cannula or the like.

Typically, compositions that are suitable for use as coatings on medical devices are applied to the surface of an implantable device by methods such as dipping, spraying or immersing. In a spraying method, the medical device is sprayed with mixed hydrogel precursors prior to gelation. In an immersion method, the medical device is immersed into the mixed hydrogel precursor while the hydrogel is forming. The choice of the method to be used is dependent on the type of device and other considerations. If desired, coating techniques can be repeated or combined to build up the polymeric coating to a desired thickness.

The biofilm-inhibiting composition coating for medical devices may be formulated to substantially prevent the colonization of device by biofilm-forming microorganisms, for example by killing and/or removing substantially all of the microorganisms on the surface of medical devices. Biofilm microorganisms include any one of the wide variety of microorganisms which form biofilms during colonization and proliferation on the surface of medical devices, including, but not limited to, gram-positive bacteria (such as Staphylococcus epidermidis), gram-negative bacteria (such as Pseudomonas aeruginosa), and/or funge (such as Candida albicans). Preferred embodiments of the invention typically target organism including Pseudomonad species, Streptococcus species, Haemophilus species, Escherichia species, Enterobacteriaceae, Proteus species, Staphylococcus species, Blastomonas, Sphingomonas, Methylobacerium and Nocardioides species as well as yeast species such as Candida albicans etc.

In accordance with an embodiment of the invention, the bactericidal vancomycin is used in combination with a CMC-DA hydrogel mixing it with the precursor solutions of the hydrogel prior to forming the coating. The vancomycin molecule will stay within the coating and release from the coating after the coating is placed at the desired site. Suitable biocidal agents that may be included in the coating include, but are not limited to, antimicrobial, antibiotics, antimyobacterial, antifungals, antivirals, and the like. Preferred antimicrobial agents include, but are not limited to, chlorhexidine, polymyxins, tetracyclines, aminoglycosides, rifampicin, bacitracin, neomycin, chloramphenicol, miconazole, quinolones, penicillins, nonoxynol 9, fusidic acid, cephalosporins, mupirocin, metronidazole, cecropins, protegrins, bacteriocins, defensins, nitrofurazone, mafenide, lincomycins, pefloxacin, nalidixic acid and combination thereof. Besides vancomycin, anti-bacterial agents may include, but are not limited to, penicillins, cephalosporins, cephamycins, carbopenems, carbopenems, monobactam, teicoplanin, macrolides, tetracyclines, aminoglycosides, chloramphenicol, sodium fusidate, azole, quinolones.

In still another embodiment of the invention, lectin and/or phosphorcholine are/is incorporated into the composition coating. Lectin and phosphorcholine are reported to help to retard the adhesion of bacterial from the surface of medical devices. While lectins and phosphocholine are used in certain embodiments of the invention, other molecules which act in an analogous manner are also suitable for use with the hydrogel coatings of the present invention.

Double-chamber syringe: The hydrogel composition can be prepared using a double-chamber syringe configuration wherein the dextran and chitosan solutions are maintained in individual chambers prior to the simultaneous introduction of the contents of each chamber to desire site of surface. Suitable syringes for this purpose are described in U.S. Pat. Nos. 4,609,371, 4,359,049, and 4,109,653, or are commercially available. The aqueous hydrogel precursors may also be conveyed though the syringe or with a variety of other common mechanical devices including, but not limited to, syringe pumps, peristaltic pumps, piston pumps, diaphragm pumps and the like.
Cell delivery: Dextran-chitosan hydrogels of the present invention may be utilized to deliver living cells to a desired site in a mammalian body. Examples of such cells include, but are not limited to, stem cells, marrow cell, bone cells, hepatocytes, keratinocytes, chondrocytes, osteocytes, endothelial cells, epithelial cells, and smooth muscles cells. Thus, the hydrogel of the present invention can be used in certain tissue engineering applications, by functioning as adhesion substrates, anchoring cells to be transplanted to affect the survival, growth and, ultimately, grafting and or anchoring of the transplanted cells to normal cellular tissue.

As was described with respect to FIGS. 7A and 7B, chondrocytes have been entrapped in this composition hydrogel by pre-mixing the cells with the hydrogel precursors to form a homogenous cell-containing solution. A cell-containing hydrogel is then formed by mixing the two aqueous hydrogel precursor with the live cells therein, and allowing gelation to occur. Such a method can be used to form cell-bearing implants.

Barrier against postoperative adhesion: The term adhesion is used to describe abnormal attachments between tissues or organs or between tissues and implants which form after an inflammatory stimulus, most commonly surgery, and in most instances produce considerable pain and discomfort. When adhesions affect normal tissue function, they are considered to be a complication of surgery. These tissue linkages often occur between two surfaces of tissue during the initial phases of post-operative repair or part of the healing process. Adhesions are fibrous structures that connect tissues or organs which are not normally joined. Common post-operative adhesions to which the present invention is directed include, for example, intraperitoneal or intraabdominal adhesions and pelvic adhesions. Adhesions may produce bowel obstruction or intestinal loops following abdominal surgery, infertility following gynecological surgery as a result of adhesion forming between pelvic structures, restricted limb motion following musculoskeletal surgery, cardiovascular complications including prolonging the operative time at subsequent cardiac surgery, infection and cerebrospinal following many surgeries, especially including spinal surgery which produces low back pain, leg pain and sphincter disturbance.
Coating for implant lens: Cataract surgery currently is a well-established ophthalmologic procedure. In cataract surgery, the diseased, clouded lens is replaced by an artificial, non-accommodating lens. Although this operation is a mature procedure, a major, severe complication of the implantation of an intra-ocular lens is the occurrence of posterior capsular opacification (PCO) caused by a proliferation of remaining epithelial cells. A polymeric coating on the surface of the implant lens is expected to retard the adhesion of remaining epithelial cell, which in turn to alleviate the posterior capsular opacification after implantation.

Typically, methods that are suitable for coating the implant lens with this composition coating include, but not limited to, dipping, spraying or immersing. All of these methods are familiar to those people who are skilled in this art. The choice of the method is dependent on the type of implant lens and other considerations. If desired, coating techniques can be repeated or combined to build up the polymeric coating to the desired thickness.

Preserving coating for food: Chitosan itself is reported to be antimicrobial, and several studies have investigated on its effect when used to coat on the surface of fruit and vegetable as a preservative due to its great biocompatibility. Typically, methods that suitable for coating the fruit and vegetable with this composition coating include, but not limited to, dipping, spraying or immersing. All these methods are familiar to those people who are skilled in this art. If desired, coating techniques can be repeated or combined to build up the polymeric coating to the desired thickness.

In an embodiment of the present invention, strawberries were treated with a CMC-DA coating through an immersing technique. After drying at room temperature, the hydrogel coating was not obviously detectable. The coating did not repel water. The coated strawberries showed an extended preservation period relative to its uncoated counterpart. The mechanism of this extended preservation time is hypothesized to be a slowed respiration rate of the fruit and/or better prevention of bacterial adhesion to the surface of the fruit.

One advantage of the dextran-chitosan hydrogel coating is that no other aqueous crosslinkers or initiators are needed which might be poisonous or add an abnormal taste to the fruit or vegetable, which would render the coating technique less valuable. A taste panel detected no-to-slight taste abnormalities for those strawberries coating with the CMC-DA composition of the present invention.

This composition coating is not only suitable for vegetables or fruit, but also can be applied to meat. The coating was applied to a meat sample in a similar manner, and the coating showed a comparable effectiveness for the meat as for the strawberries.

In further embodiments of the invention, this edible hydrogel coating can serve as carriers for a wide range of food additives, including various antimicrobials that can extend product shelf-life and reduce the risk of pathogen growth on food surface. In this embodiment, antimicrobials which are generally recognized as safe may be incorporated into processed meat formulations, applied as dipping solution or sprayed on the surface of the sample.

The examples presented herein describe representative embodiments of the invention and are in no way intended to limit the range of embodiments encompassed by the present disclosure. A person skilled in the relevant arts may make many variations and modifications of the hydrogels discussed herein without departing from the spirit and scope of the invention as defined in the claims below.

1. A composition, comprising a hydrogel formed by a process including the step of mixing a first aqueous solution including a chemically-modified water soluble dextran having a plurality of aldehyde groups with a second aqueous solution including a chemically-modified water-soluble chitosan that is at least partially deacetylated, whereby said dextran spontaneously cross-links with said chitosan.

2. The composition of claim 1, wherein said chitosan has a plurality of carboxymethyl groups.

3. A composition, comprising a chemically-modified water-soluble dextran having aldehyde groups, said dextran being cross-linked with a chemically-modified water-soluble chitosan that is at least partially deacetylated.
